(19) United States
(12) Patent Application Publication
(10) Pub. No.: US 2013/0267774 A1
(43) Pub. Date: Oct. 10, 2013

(54) GASTRIC BAND WITH ELECTRIC STIMULATION

(71) Applicant: Allergan, Inc., Irvine, CA (US)

(72) Inventors: Janel A. Birk, Oxnard, CA (US); Sean Snow, Carpinteria, CA (US)

(21) Appl. No.: 13/909,631

(22) Filed: Jun. 4, 2013

Related U.S. Application Data
(63) Continuation of application No. 12/973,566, filed on Dec. 20, 2010, now abandoned.
Application No. 12/973,566 is a continuation-in-part of application No. 12/853,498, filed on Aug. 10, 2010.
(60) Provisional application No. 61/237,881, filed on Aug. 28, 2009.

(51) Int. Cl. A61F 5/00

(52) U.S. Cl. CPC A61F 5/0026; A61F 5/005
USPC 600/37

(57) ABSTRACT

Apparatus and methods for stimulating one or more nerves by incorporating a gastric band system and an electrical stimulation component. Stimulation electrodes on the gastric band may be used to stimulate the vagal nerve and/or splanchnic nerve, which may inhibit the patient's appetite and/or control obesity. The gastric band may have an inflatable member for adjusting a stoma size. The stimulation electrodes may be mounted on the inflatable member. The system may include a controller including a pressure sensor for monitoring the hydraulic pressure within the inflatable inner member and for controlling the stimulation component.

### RELATED APPLICATIONS

This application is a continuation of U.S. patent application Ser. No. 12/973,566, filed Dec. 20, 2010, which is a continuation-in-part of U.S. patent application Ser. No. 12/853,498, filed Aug. 10, 2010, which claims priority to U.S. Provisional Patent Application No. 61/237,881, filed Aug. 28, 2009, the disclosures of which are hereby incorporated in their entirety herein by reference.

### FIELD

The present invention relates, in general, to devices and methods for controlling obesity, and, more particularly, to a gastric band or gastric band assembly/system that provides ongoing adjustment of stoma size in a patient in conjunction with electrical stimulation of the stomach.

### BACKGROUND

Severe obesity is an increasingly prevalent chronic condition that is difficult for physicians to treat in their patients through diet and exercise alone. Gastrointestinal surgery is used by physicians to treat patients who are severely obese and cannot lose weight by traditional means or who suffer from serious obesity-related health problems. Generally, gastrointestinal surgery promotes weight loss by restricting food intake, and more specifically, by creating a narrow passage or “stoma” from the upper part of the stomach into the larger lower part, which reduces the amount of food the stomach can hold and slows the passage of food through the stomach. Initially, the stoma was of a fixed size, but physicians have more recently determined that the procedure is more effective if the stoma can be adjusted to alter its size.

One of the more commonly used of these purely restrictive operations for obesity is an adjustable gastric banding (AGB) system. In an exemplary AGB procedure, a hollow band (i.e., a gastric band) made of silicone elastomer is placed around the stomach near its upper end, creating a small pouch and a narrow passage (i.e., a stoma) into the rest of the stomach. The band is then inflated with a saline solution by using a non-coring needle and syringe to access a small port that is placed underneath the skin. To control the size of the stoma, the gastric band can be tightened or loosened over time by the physician or another technician extracorporeally by increasing or decreasing the amount of saline solution in the gastric band via the access port to change the size of the stoma.

Providing fine adjustments of the gastric band after initial stoma sizing has proven to be a significant improvement in the adjustable gastric banding procedure. However, there is an ongoing difficulty in determining when to further adjust the gastric band and how much to increase or decrease the band's size or diameter to achieve a desired stoma size. Numerous gastric bands have been developed to allow a physician or other technician to adjust an implanted gastric band. In general, these gastric band systems include a sensor for measuring or determining parameters associated with the patient and in response, the physician or technician acts to adjust the volume of fluid in the gastric band based on the patient's parameters. For example, one adjustable gastric band system determines when the pressure in a patient's stomach exceeds a pre-set limit and provides an alarm to an external control device. A doctor or other operator then responds by loosening the gastric band by removing an amount of fluid from the gastric band via the access port and the fill line.

In another gastric band system, disclosed in Gertner, U.S. Patent Application Pub. No. 2006/0089571, gastric bands may operate in conjunction with electrical stimulation of the stomach. In one embodiment, a transgastric fastening assembly serves to reduce the volume of the stomach as well as provide for electrical stimulation. An electrical signal runs through electrodes in the transgastric fastener assembly to possibly alter the contraction patterns of the stomach or to electrically create a feeling of satiety in addition to reducing the volume of the stomach and creating a restriction to flow in the stomach.

Due to certain limitations of existing technologies, there remains a need for an improved gastric band system, and associated adjustment methods, for providing improved adjustments to the size of a stoma in a patient being treated for obesity-related health issues.

### SUMMARY

The present invention provides a gastric band or band system that incorporates an electrical stimulation system. The resulting implantable medical device provides the treatment of a gastric band with the treatment of functional electrical stimulation of the nervous system.

In one embodiment, a gastric band includes any number of devices in contact with the upper surfaces of the stomach, such as the cardia region. The stomach surfaces may provide adequate surface area for holding stimulation electrodes. When current passes through the electrodes the vagal nerve and/or splanchnic nerve are stimulated, which may advantageously inhibit the patient's appetite. In one embodiment, a sensor may be used in conjunction with the gastric band to sense a change in pressure exerted on the gastric band by the stomach and/or a change in the volume of the fluid within the inflatable members of the gastric band. The sensor may transmit information related to the changes to the processor, which in turn, may determine that the stimulation electrodes should stimulate the stomach nerves (e.g., the splanchnic nerve) and may choose an appropriate stimulation wave (e.g., a stimulation signal or wave having an appropriate current, voltage, frequency, wavelength, power, time duration, etc.) for sending to the electrode(s) contacting the stomach.

In another embodiment, a method of inhibiting appetite and promoting weight loss includes determining that an electrical stimulation of a nerve is required, passing a current through an electrode to stimulate the nerve and confirming that the nerve stimulation was successful.

In another embodiment, a method of inhibiting appetite and promoting weight loss includes sending an electric signal to a stimulation electrode, determining if the patient responds with nausea, and/or fullness, and changing the signal and/or destination electrodes if the patient responds feeling nauseous or still hungry, and updating the patient record accordingly.

### BRIEF DESCRIPTION OF THE DRAWINGS

Features and advantages of the present invention will become appreciated as the same become better understood with reference to the specification, claims, and appended drawings wherein:

FIG. 1 is a schematic view of a gastric band system in accordance with one or more embodiments described herein.

FIG. 2 is a top view of a gastric band in accordance with one or more embodiments described herein.

FIG. 3 is a cross-sectional view of the gastric band shown in FIG. 2 taken along line 3-3 in accordance with one or more embodiments described herein.

FIG. 4 is a top view of an encircling portion of an inflatable shell of the gastric band shown in FIG. 1 in accordance with one or more embodiments described herein.

FIG. 5 is a side view of the encircling portion of the inflatable shell of the gastric band shown in FIG. 1 in accordance with one or more embodiments described herein.

FIG. 6 is a cross-sectional top view of the inflatable shell shown in FIG. 5 taken along line 6-6 in accordance with one or more embodiments described herein.

FIG. 7 is a close up cross-sectional view of a convolution point of the inflatable shell shown in FIG. 6 taken in area 7 in accordance with one or more embodiments described herein.

FIG. 8 is a cross-sectional view of the inflatable shell shown in FIG. 4 taken along line 8-8 showing the relative thickness of an electrode in accordance with one or more embodiments described herein.

FIGS. 9 and 10 are cross-sectional views of the encircling portion of the inflatable shell shown in FIG. 4 taken along line 9-9 and 10-10, respectively, in accordance with one or more embodiments described herein.

FIG. 11 is a portion of a gastric band including an electrode and a wire in accordance with one or more embodiments described herein.

FIG. 12 is a three-dimensional view of a controller in accordance with one or more embodiments described herein.

FIG. 13 is a block diagram of a controller in accordance with one or more embodiments described herein.

FIG. 14 is a flow chart of a method of stimulating a nerve by using an electrode in accordance with one or more embodiments described herein.

FIG. 15 is a flow chart of a method of sending a signal to an electrode in accordance with one or more embodiments described herein.

FIG. 16 is a flow chart of a method of determining optimal nerve stimulation in accordance with one or more embodiments described herein.

The figures included herein are exemplary and might not include the same features as when compared to other figures. For example, certain features of a figure may be omitted in a separate, related figure for clarity and ease of understanding.

### DETAILED DESCRIPTION

The present invention provides a gastric band or band system that incorporates a functional electrical stimulation system. The resulting implantable medical device provides the treatment of a gastric band with the treatment of functional electrical stimulation of the nervous system.

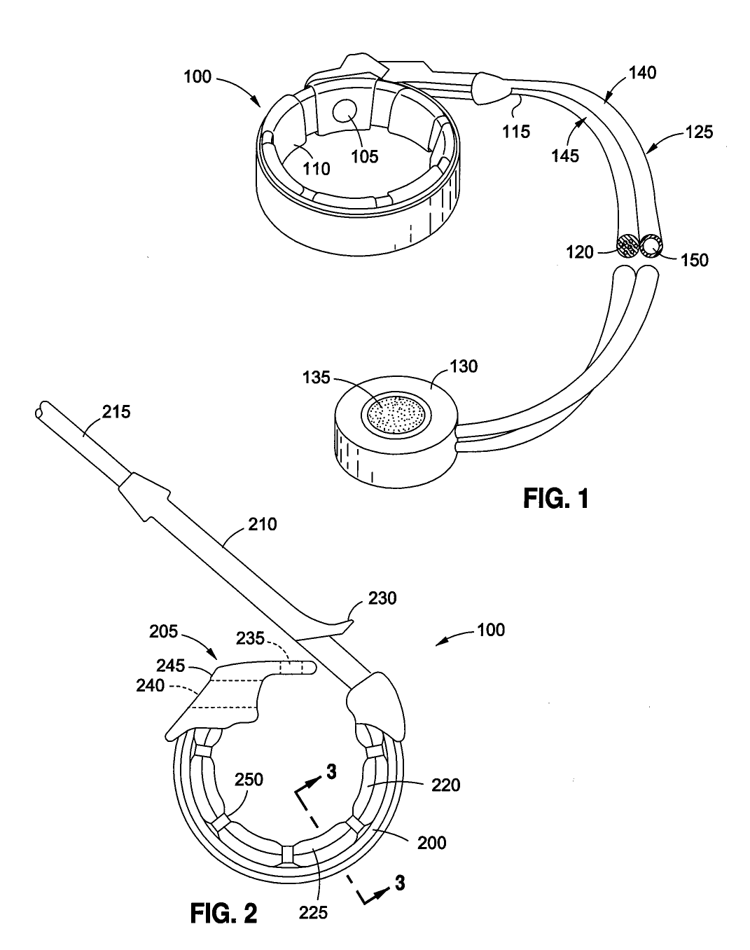

As shown in FIG. 1, a gastric band electrical stimulation system includes a gastric band 100 on which is placed one or more electrodes 105, a band member 110, and a controller 130. In one embodiment, the gastric band 100 includes a generally circular band member 110 to which a flexible cord 115 tangentially attaches. The flexible cord 115 may include a tubular conduit 125. The tubular conduit 125 may be sub-divided into two generally parallel and individually tubular housings. A housing 140 may allow fluid 150 to travel from an access port 135 to the gastric band 100 and from the gastric band 100 to the access port 135, thereby inflating the gastric band 100 when the fluid 150 is added, and deflating the gastric band 100 when the fluid 150 is removed. A housing 145 may allow lead wires 120 to travel from the controller 130 to the gastric band 100 (e.g., the one or more electrodes 105). In one embodiment, the lead wires 120 and the fluid 150 are separated by the walls of the housings 140 and 145.

The stimulation electrodes 105 are positioned on an inner surface of the band member 110. The lead wires 120 located within the housing 145 may function to carry a signal in the form of electric current from a power source located inside the controller 130 to the electrodes 105. When implanted, the band member 110 surrounds and contacts an upper region of the stomach, such as the stoma or cardia. The inner surface may be relatively solid, or may be an inflatable ring used for adjusting the amount of constriction on the stoma. Several prominent nerves extend through the upper portions of the stomach (e.g., the vagal nerve, the splanchnic nerve and the like), and stimulating them by introducing a current from the electrodes 105 may induce feelings of satiety (fullness) or nausea in a patient, either of which inhibits the appetite.

A number of different gastric bands are available today, and the present disclosure may be used with any number of these as well as others not yet known or marketed. For example, a preferred gastric band for use is sold under the name LAP-BAND® Adjustable Gastric Banding System by Allergan, Inc. of Irvine, Calif., and is designed to be placed laparoscopically (via small incisions in the abdomen, usually 0.5-1.5 centimeters in length). An inflatable band is placed around the top portion of the patient's stomach, creating a small pouch that limits or reduces food consumption. The LAP-BAND® System is adjustable, which means that the inflatable band can be tightened or loosened to help the patient achieve a level of satiety while maintaining a healthy diet, supporting a patient's long-term weight loss success. Other possible gastric bands are adjustable electromechanically without hydraulics, and still others may have a fixed-size with no adjustment.

The controller 130 may include an access port 135 for adjusting the gastric band 100 percutaneously. The controller 130 incorporates a power source and circuitry for controlling and delivering precise pulses of electrical current to the electrodes 105 on the inner surface of the band member 110. In one embodiment, the external case of the controller 130 is conductive and functions as a return electrode for the gastric band electrodes 105.

FIG. 2 is a top view of the gastric band 100 of FIG. 1. In this embodiment, the gastric band 100 may be inflatable or deflatable with a fluid to adjustably constrict and release the stoma. The gastric band 100 has a body portion 200 and an inflatable portion or shell 225. The body portion 200 has a head end 205 and a tail end 210. The head end 205 may include a buckle 240 with a pull-tab 235. The tail end 210 may include a belt tab 230. Upon insertion of the tail end 210, including a fill tube 215 through the buckle 240, the tail end 210 may be drawn through the buckle 240 until the belt tab 230 catches on the exit side 245. In this position, the gastric band 100 is releasably locked in a closed loop position and secured by the buckle 240 and the belt tab 230.

The fill tube 215, which may be a tube having a single lumen (not shown) coextensive therewith, and may be connected to an end of the gastric band 100. In FIG. 2, the fill tube 215 is shown attached to the tail end 210 and is in fluid communication with the inflatable shell 225. It will be apparent to one of ordinary skill in the art that other arrangements of the fill tube 215 can be made including attachment to the head end 205 without departing from the scope of the present disclosure.

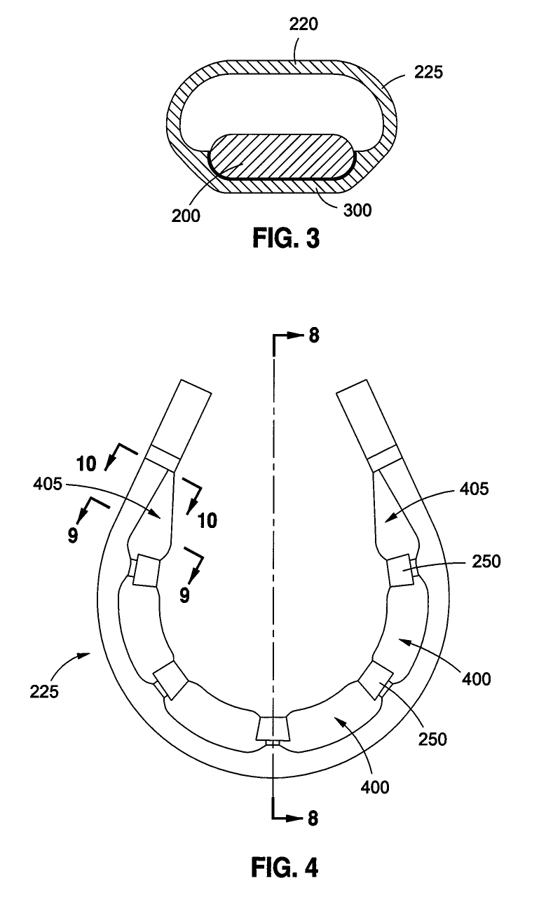

Turning to FIG. 3, which may be a cross sectional view of FIG. 2 taken along line 3-3, the inflatable shell 225 may be formed to receive the body portion 200. The inflatable shell 225, in one embodiment may be substantially coextensive with the body portion 200, as shown in FIG. 2. The inflatable shell 225 may include an inner stomach-facing surface 220 that forms a stoma when placed around the stomach. FIG. 3 further illustrates the outer surface 300.

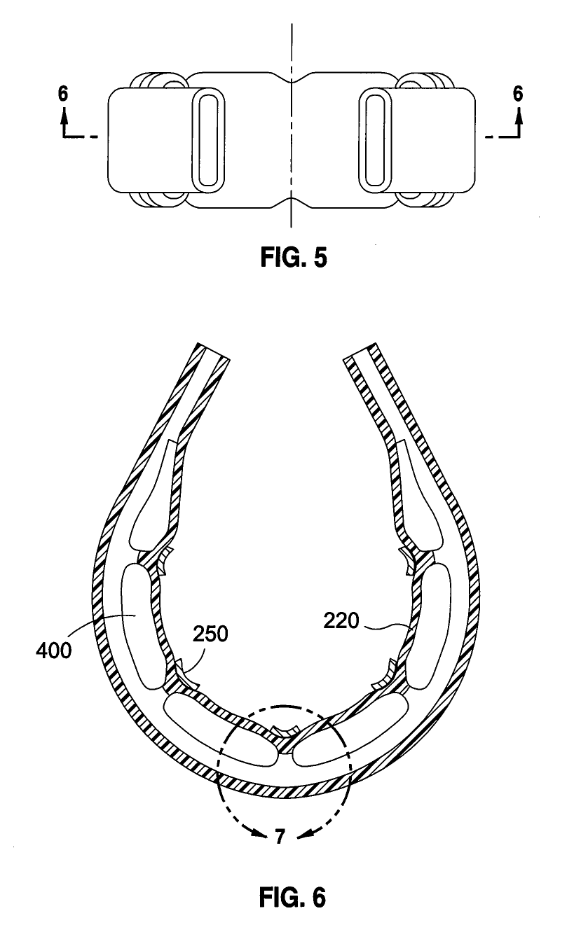

FIG. 4 shows a top view of the inflatable shell 225 of the gastric band 100. As shown in FIG. 4, one or more electrodes 250 may be placed between two adjacent chambers 400. In one embodiment, the number of electrodes 250 may be one less than the number of chambers 400 and end chambers 405 of the inflatable shell 225. The end chambers 405 (i.e., the chambers closest to the head end and tail end, respectively) may be, in one embodiment, shaped differently than the other chambers (e.g., chambers 400) for reasons discussed later in this disclosure. FIG. 5 is a side view of the inflatable shell 225 of FIG. 4.

In one embodiment, the electrodes 250 may be placed on the outside surface of the gastric band 100, such that electrical impulses received and transmitted by the electrodes 250 may traverse through the inflatable shell 225 and the fluid therein to stimulate the stoma (and therefore, also stimulating the splanchnic, vagal or other nerves) to increase the feeling of satiety in the patient.

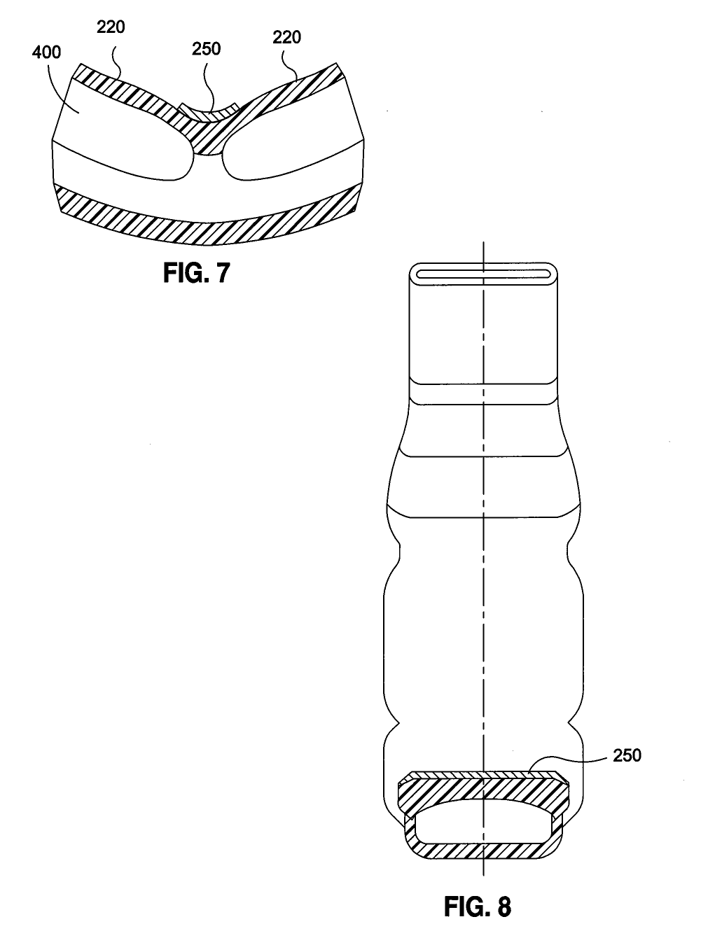

FIG. 6 illustrates a cross-sectional top view of the inflatable shell 225 shown in FIGS. 4 and 5. In FIG. 6, one side of the inflatable shell 225 is depicted with attached electrodes 250 at each of the notches separating each of the chambers 400. Additionally, each electrode 250 may be layered on top of the inner stomach-facing surface 220 in direct contact with the stoma. FIG. 7 is a close up cross-sectional view of a convolution point of the inflatable shell of FIG. 6 taken in area 7.

FIG. 8 is a cross-sectional view of the inflatable shell 225 of FIG. 4 taken along line 8-8 showing the relative thickness of the electrode 250. In one embodiment, the electrode 250 nearly spans the entire width of the inflatable shell 225 of FIG. 4. Alternatively, the electrode 250 may be configured to be much smaller than shown. For example, the electrode 250 may span half the width or a quarter of the width of the inflatable shell 225 shown in FIG. 4. A smaller electrode may provide different advantages such as lower cost.

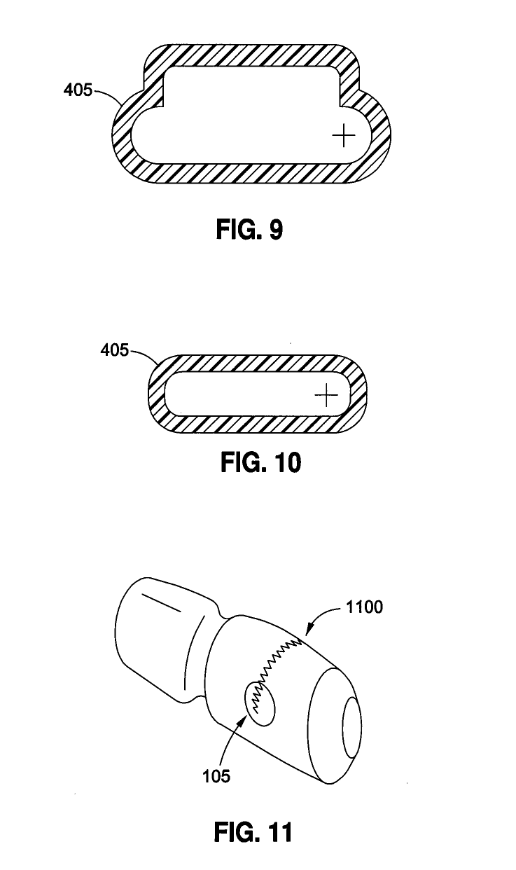

FIGS. 9 and 10 are cross-sectional views of the encircling portion of the inflatable shell 225 shown in FIG. 4 taken along lines 9-9 and 10-10, respectively. More specifically, the two cross-sectional views depicted in FIGS. 9 and 10 illustrate two different cross-sections within the chamber 405. In one embodiment, the chambers 405 shown in FIGS. 9 and 10 are the chambers located closest to the head end 205 and the tail end 210, respectively. FIG. 10 depicts the opening of the chambers 405 to be much smaller as it tapers towards the two respective ends, while FIG. 9 depicts the opening to be larger in comparison to the opening shown in FIG. 10. In one embodiment, the design of the tapering ends of the chambers 405 advantageously provide comfort to a patient when the gastric band 100 forms a generally circular band as the two ends form a closed loop. The openings of the other chambers 400 may be similar in size to the opening depicted in FIG. 4.

FIG. 11 is a portion of a gastric band including an electrode 105 and a wire 1100. In one embodiment, the electrode 105 may be a platinum (Pt) and/or an iridium (Ir) disk-activated electrode. In another embodiment, the electrode 105 may be attached to the gastric band 100 in one of a number of different ways. For example, the electrode 105 may be glued permanently on the gastric band 100, embedded on the surface of the gastric band 100, constrained by over-sheath of a conductive element such as silicone, and the like. The wire 1100 may be constructed out of a conductive material such as gold. Moreover, the wire 1100 may be stress-relieved and may be resistant to work-hardening. While this example may be based on the electrode 105 as shown in FIG. 1, its applicability is not limited to the example, and in another embodiment, may apply to electrode(s) 250 as shown in other figures, such as FIG. 2. In another embodiment, the wire 1100 may be encapsulated by an insulator.

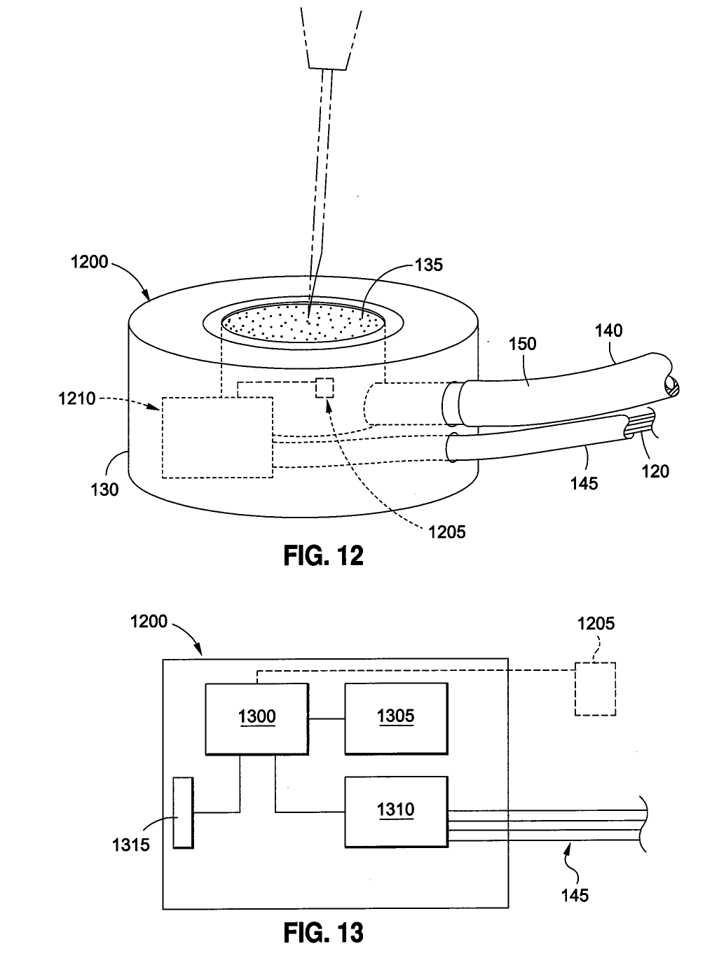

FIG. 12 is a three-dimensional view of a controller in accordance with one or more embodiments described herein. In one example, the controller of FIG. 12 may be the controller 130 shown in FIG. 1. The controller 130 may include a casing 1200. In one embodiment, the casing 1200 may be configured to include an access port 135 and further include an opening to receive wires 120 and band adjustment fluid 150 via the housings 145 and 140, respectively. Within the casing 1200, internal circuitry 1210 may be coupled to a sensor 1205 located in a position to allow detection of various properties of or within the tubular conduit 125 and the fluid 150 therein (e.g., pressure, volume and the like). The fluid 150 may be added or removed percutaneously via the access port 135 by using a needle or other skin penetrating device. In addition, the controller 130 may also house a portion of the flexible housings 140 and 145, the wires 120 and the fluid 150. In one embodiment, the controller 130 may be attached inside the patient's body according to methods known in the art.

FIG. 13 is a block diagram of the internal circuitry 1210 of FIG. 12. FIG. 13 may include a processor 1300, a memory 1305, a power source 1310 and a transceiver 1315. The processor 1300 may cause the controller 130 and/or the gastric band 100 to function in accordance with one or more embodiments herein by executing instructions stored in the memory 1305. In one example, the processor 1300 may be coupled to the transceiver 1315 and may receive instructions from a computer located externally to the patient through signals received by the transceiver 1315. These signals may request the processor 1300 to command the power source 1310 to send an electrical pulse to one or more electrodes 105 or 250. In another example, the processor 1300 may be coupled to the sensor 1205, and based on input received from the sensor 1205, such as the amount of pressure the gastric band 100 is exerting or a volume level of the fluid 150, the processor 1300 may determine that it is appropriate to send an electrical pulse to electrodes 105 or 250 based on a predetermined pressure and/or volume level threshold. The power source 1310 may be a battery and/or another device configured to generate electric signals. The power source 1310 may be connected to the wires 120 and configured to transmit electrical pulses to the electrodes (e.g., the electrodes 105 or 250) via the wires 120. In addition to storing instructions, the memory 1305 may be used to store other information such as waveform types, commands received, results of the sending of the electrical signals and the like. The memory 1305 may be a physical storage medium (e.g., Read-Only memory, Random Access memory, Flash, Electrically Erasable Programmable Read-Only Memory, etc.) coupled to the processor 1300, among other components of the controller 130.

In one embodiment, the sensor 1205 may further be used to obtain physiological information and in turn operate the gastric band 100 and the one or more electrodes 250. For example, the sensor 1205 may be a pressure sensor for monitoring the hydraulic pressure within the gastric band 100. In another example, the sensor 1205 may be used to ascertain the amount of fluid within the band member 110 and/or the amount of fluid in the conduit 125.

In one alternative, the hydraulic pressure may be measured within the lumen of the tubular conduit 125 or a sensor (e.g., sensor 1205) may be incorporated into the gastric band 100 itself, with a wire or wireless interface to the controller 130 (not shown). The pressure information can be used diagnostically by the physician or may be used to control the electrical stimulation.

In one mode of operation, the gastric band (e.g., gastric band 100) functions normally with adjustments made to the level of constriction through fluid transfer, either by percutaneous addition through the access port 135 or by a needle-free telemetric system that utilizes an implantable fluid pump(s) and reservoir(s).

Referring back to FIG. 1, the electrodes 105, in one embodiment, may serve as the source or current return, i.e., as the anode or cathode. Each electrode 105 receives current from or transmits current to the controller 130 via insulated leads or wires 120. The wires 120 pass along the flexible cord 115 (e.g., in the housing 145) parallel to a tubular conduit 140 having a lumen for flow of fluid, typically saline. Fluid may be added or removed from within a balloon on the interior of the gastric band 100 to adjust constriction of the stomach. The wires 120 may be encompassed by a polymer jacket that is adhered to or molded with the housing 145. The wires 120 are formed into a geometry which provides strain relief and resists fracturing.

The functional electrical stimulation system operates by applying a precisely-controlled voltage across the source and return electrode. In one embodiment, the two electrodes (e.g., electrodes 105 or 250) may both be on the inner surface of the band member 110, or one may be remote, such as on the controller 130. As such, in the following description in this paragraph, the description related to the electrodes may refer to the electrodes 105 or 250, and/or any suitable device that may act as an electrode on the controller 130. The potential difference across the electrodes 105 or 250 creates a current flow through the tissue in contact with the electrodes of a desired duration and amplitude. The type of electrodes and signals used varies depending on the desired effect. Choices include monopolar or bipolar delivery, monophasic and biphasic charge pulses, interphase intervals, active and passive charge recovery, variable and fixed frequency, symmetric and asymmetric phases, and various waveform shapes. In one embodiment, different signals may be sent to different electrodes. For example, a first signal may be sent to certain electrodes to stimulate the splanchnic nerve, while a second signal may be sent to certain electrodes to stimulate the vagal nerve. As current flows through the tissue, the neurons located therein experience depolarization and, ultimately, activation. The action potentials are then conducted by the neurons to the regions of the body which induce feelings of satiety or nausea.

The functional electrical stimulation pulses may be programmed to follow a number of different protocols. For example, the pulses may be activated on a timing system, such as on a daily schedule at documented times when the patient experiences hunger cravings. In one embodiment, timing system information may be stored in the memory 1305 of FIG. 13, and may adjusted by an external computer via transmittal of commands through the transceiver 1315. Alternatively, the pulses may be controlled on the basis of feedback from the band pressure monitoring system. For instance, pressure variations within the gastric band 100 may indicate the ingestion of food, which acts to raise the pressure within the gastric band 100. The controller 130, in this embodiment, may be programmed to detect such pressure changes and fire the stimulation pulses to thereby reduce the patient's appetite at the time of eating.

The electrodes 105 of FIG. 1 and/or electrodes 250 of FIG. 2 are preferably formed of thin plates of suitably conductive and biocompatible material, such as platinum (Pt), iridium (Ir), Pt/Ir alloy, tantalum, etc. The electrodes (e.g., electrode 105 or 250) may be arranged linearly on the surface of the band member 110 or in a two-dimensional pattern. In one embodiment, the electrodes (e.g., electrode 105 or 250) may be formed on the troughs of the inner circumference of the inflatable inner member (e.g., as shown in FIG. 4). Alternatively, the electrodes (e.g., electrode 105 or 250) may be formed on the peaks of the inner circumference of the inflatable inner member (not shown in FIG. 4). In another embodiment, the electrodes (e.g., electrode 105 or 250) may be formed on both the peaks and troughs of the inner circumference of the inflatable inner member (not shown in FIG. 4). The placement of the electrodes may, in one embodiment, be configured to be proximal to the nerve or specific stomach region that the electrode is intended to stimulate.

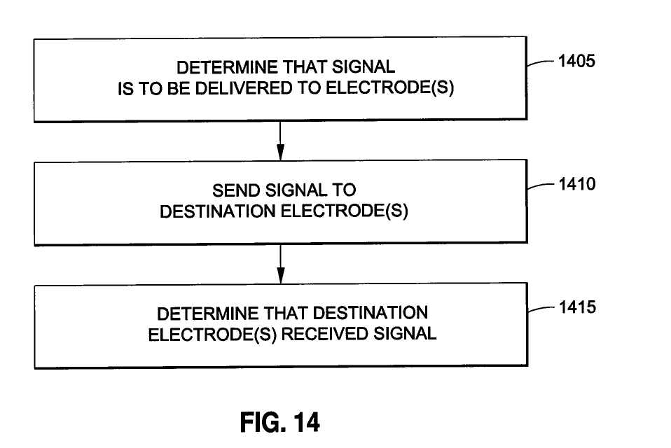

FIG. 14 illustrates an example of a method in accordance with one or more embodiments described herein. In one example, the instructions for the method may be stored in a memory, such as the memory 1305 of FIG. 13. The execution of the method of FIG. 14 may be performed by a number of different elements including, for example, the processor 1300 and the power source 1310. At step 1405, the processor 1300 may determine that an electronic signal is to be delivered to an electrode (e.g., electrode 105 or 250) to stimulate a nerve located in the stomach region of the patient. The determination may be triggered by one or a combination of a number of different ways, not limited to, but including receiving instructions from a computer located externally to the patient, or detecting that it is time that the patient typically eats (e.g., 6:00 PM or 8:00 AM), and/or triggered by feedback from the band pressure monitoring system (e.g., pressure variations within the gastric band 100 may exceed a predetermined threshold which indicates the ingestion of food). At step 1410, the processor 1300 may instruct a power source (e.g., power source 1310) to send an electrical signal to the destination electrode (e.g., one or more of the electrodes 105 or 250). At step 1415, the processor 1300 may receive indication that the destination electrode (e.g., electrode 105 or 250) received the signal. In one embodiment, indication of the receipt of signal may be assumed when it is detected that the patient is no longer ingesting food (e.g., where a sensor 1205 detects that the pressure is no longer increasing). In another aspect of the same or different embodiment, a return signal may be transmitted to the power source 1310 by the electrode (e.g., electrode 105 or 250).

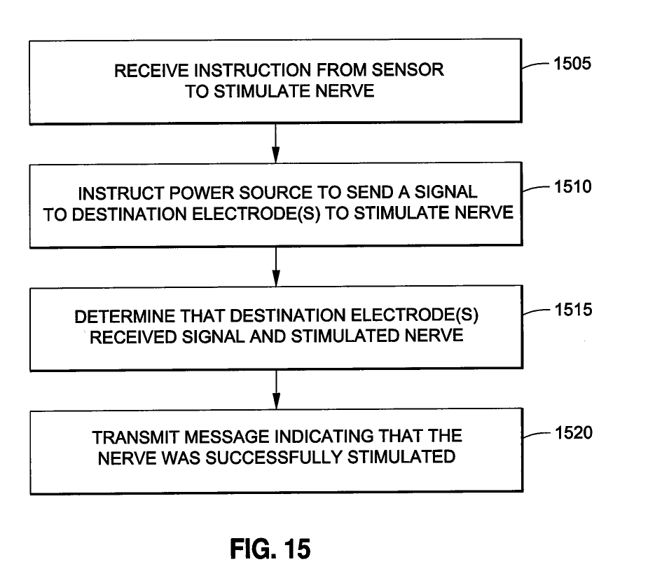

FIG. 15 illustrates a method of sending a signal to an electrode (e.g., electrode 105 or 250). In one example, the instructions for the method may be stored in a memory, such as the memory 1305 of FIG. 13. The execution of the method of FIG. 14 may be performed by a number of different elements including, for example, the processor 1300, the sensor 1205, the transceiver 1315 and/or the power source 1310. At step 1505, the sensor (e.g., the sensor 1205) may transmit a signal to the processor (e.g., the processor 1300) to begin nerve stimulation. The sensor (e.g., the sensor 1205) may be programmed to send the signal in response to detecting one of a numerous criteria being met. For example, if the sensor (e.g., sensor 1205) detects a change in fluid level, it may ascertain whether the fluid level increased above a pre-determined threshold or decreased below a pre-determined threshold. Similarly, if the sensor (e.g., sensor 1205) detects a change in pressure, it may ascertain whether the pressure increased above a pre-determined threshold or decreased below a pre-determined threshold. At step 1510, the processor (e.g., processor 1300) may instruct the power source (e.g., power source 1310) to send a signal to a destination electrode (e.g., electrode 105 or 250) to stimulate the nerve. In addition, the processor (e.g., processor 1300) may further instruct which type of signal to send. At step 1515, the processor (e.g., processor 1300) may receive indication that the destination electrode (e.g., electrode 105 or 250) successfully received the signal and that the nerve was stimulated. At step 1520, the processor (e.g., processor 1300) may instruct the transceiver (e.g., transceiver 1315) to transmit a message to a computer system external to the patient that the nerve was successfully stimulated.

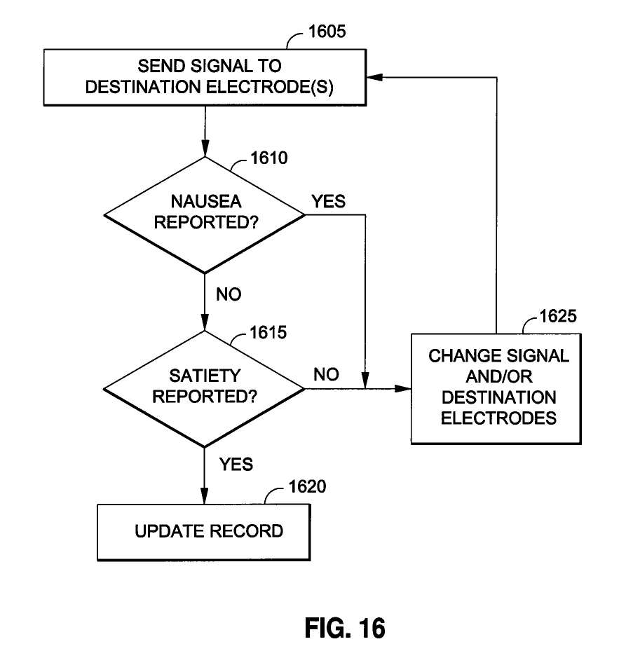

FIG. 16 illustrates a method of determining optimal nerve stimulation in accordance with one or more embodiments described herein. In one example, the instructions for said method may be stored in a memory, such as the memory 1305 of FIG. 13. The execution of the method of FIG. 16 may be performed by a number of different elements including, for example, the processor 1300, the sensor 1205, the transceiver 1315 and/or the power source 1310. At step 1605, a power source (e.g., power source 1310) may send a signal to a destination electrode (e.g., electrode 105 or 250) to stimulate the nerve. In response, a determination may be made at step 1610 to ascertain whether the patient feels nausea (e.g., if the patient reports nausea and/or vomiting, based on a patient's response when asked if he/she feels nauseous and the like). If so, an external computer may transmit a signal to a transceiver (e.g., transceiver 1315) to alert the power source to stop sending the signal causing the patient to suffer from the symptoms of nausea. However, if the patient does not report nausea at step 1610, a determination may be made at step 1615 to ascertain whether the patient feels satiety, fullness or not hungry. If so, the patient's record is updated at step 1620 to reflect that the signal transmitted did not result in nausea and further resulted in the patient feeling full and not hungry. However, if the patient reports nausea at step 1610 or that the patient is still hungry or not full at step 1615, a different signal and/or destination electrodes (e.g., electrode 105 or 250) may be selected at step 1625 before the newly selected signal is sent and/or newly selected destination electrodes (e.g., electrode 105 or 250) receives the signal. Accordingly, an optimal signal and/or electrodes may be determined and utilized any time the patient's nerves are to receive stimulation.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, references may have been made to patents and printed publications in this specification. Each of the above-cited references and printed publications are individually incorporated herein by reference in their entirety.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An implantable gastric band for introducing an electrical signal to a splanchnic nerve of a patient, the implantable gastric band configured to be used to treat obesity, comprising:
a body having a head end including a buckle, and a tail end including a belt tab;
an inflatable member connected to the body between the head end and the tail end;
a conduit for carrying fluid, the conduit configured to be fluidly coupled to the inflatable member;
an electrode attached to the body or the inflatable member, the electrode configured to introduce the electric signal to the splanchnic nerve; and
a wire connected to the electrode, the wire being encapsulated by an insulator.

2. The gastric band of claim 1, wherein the electrode is further configured to introduce an electric signal to a vagal nerve.

3. The gastric band of claim 2, further comprising a sensor coupled to the wire, the sensor configured to obtain physiological information related to the patient.

4. The gastric band of claim 3, wherein the inflatable member is configured to inflate or deflate based on the physiological information obtained by the sensor.

5. The gastric band of claim 3, wherein the electrode is further configured to introduce the electric signal to the splanchnic nerve based on the physiological information obtained by the sensor.

6. The gastric band of claim 3, wherein the electrode is further configured to introduce the electric signal to the vagal nerve based on the physiological information obtained by the sensor.

7. A method of stimulating a splanchnic nerve to control an appetite of a patient, comprising:
determining a first electric signal to be transmitted to an electrode for stimulating the splanchnic nerve;
transmitting the first electric signal to the electrode to stimulate the splanchnic nerve; and
determining that the electrode successfully received the first electric signal.

8. The method of claim 7, further comprising:
determining a second electric signal to be transmitted to an electrode for stimulating the vagal nerve;
transmitting the second electric signal to the electrode to stimulate the vagal nerve; and
determining that the electrode successfully received the second electric signal.

9. The method of claim 7, wherein determining the first electric signal to be transmitted is based on physiological information obtained by a sensor inside a body of the patient.

10. The method of claim 7, further comprising transmitting a message to a computer outside the body of the patient that the electrode received the first electric signal.

11. The method of claim 7, further comprising:
determining if nausea was reported after transmitting the first signal to the electrode;
selecting a different signal to be transmitted to the electrode in response to determining that nausea was reported; and
transmitting the different signal to the electrode.

12. The method of claim 7, further comprising:
determining if satiety was reported after transmitting the first signal to the electrode;
selecting a different signal to be transmitted to the electrode in response to determining that satiety was not reported; and
transmitting the different signal to the electrode.

13. A gastric band system with electrical stimulation, comprising:
an implantable gastric band having an inflatable member and a stimulation electrode on an inner circumference of the implantable gastric band;
an implantable controller coupled to the stimulation electrode, the implantable controller having a power source and circuitry for sending electrical pulses;
a flexible cord extending from the gastric band to the implantable controller, the flexible cord enclosing electric wires connecting the stimulation electrode to the implantable controller; and
a fluid conduit in communication between the inflatable member and an implanted reservoir, the fluid conduit extending in parallel with the flexible cord,
wherein the stimulating electrode is configured to contact a patient's stomach region and deliver electric stimulation to a splanchnic nerve in response to receiving an electric pulse from the power source, the electric pulse traversing the electric wires.

14. The system of claim 13, wherein the stimulating electrode further delivers an electric stimulation to a vagal nerve in response to receiving the electric pulse from the power source.

15. The system of claim 13, wherein the gastric band further includes an adjustable circumference.

16. The system of claim 13, wherein the stimulation electrode is mounted on an inner surface of the inflatable member.

17. The system of claim 13, wherein the implantable controller includes a return electrode for the stimulation electrode.

18. The system of claim 13, wherein the inflatable member has an uneven inner circumference having inwardly-directed troughs and peaks on which are mounted a series of the stimulation electrodes in the troughs.

19. The system of claim 13, wherein the inflatable member has an uneven inner circumference having inwardly-directed troughs and peaks on which are mounted a series of the stimulation electrodes in the peaks.

20. The system of claim 13, further comprising a sensor coupled to the implantable controller, the sensor being configured to obtain physiological information and to feed the physiological information to the circuitry for operating either a gastric band size adjustment and/or the stimulation electrode.

21. The system of claim 20, wherein the stimulation electrode is mounted on the inner surface of the inflatable member, and the sensor includes a pressure sensor for monitoring the hydraulic pressure within the inflatable member.

22. A gastric band system with electrical stimulation, comprising:
an implantable gastric band having an inflatable inner member with an uneven inner circumference having inwardly-directed troughs and peaks on which are mounted a series of stimulation electrodes in the peaks; and
an implantable controller including a power source and circuitry for sending electrical pulses to the stimulation electrodes.

23. The system of claim 22, wherein the stimulation electrodes are configured to stimulate a splanchnic nerve.

24. The system of claim 23, wherein the stimulation electrodes are configured to stimulate a vagal nerve.
